(19) United States
(12) Patent Application Publication
(10) Pub. No.: US 2026/0097032 A1
(43) Pub. Date: Apr. 9, 2026

(54) PHARMACEUTICAL COMPOSITION, AND PREPARATION METHOD THEREFOR AND USE THEREOF

(71) Applicant: GENFLEET THERAPEUTICS (SHANGHAI) INC., Shanghai (CN)

(72) Inventors: Yuanyuan XIE, Shanghai (CN); Lina QI, Shanghai (CN); Can HUANG, Shanghai (CN); Jiong LAN, Shanghai (CN); Qiang LU, Shanghai (CN)

(21) Appl. No.: 19/113,377

(22) PCT Filed: Sep. 20, 2023
(86) PCT No.: PCT/CN2023/120042

§ 371 Date: Mar. 19, 2025

(30) Foreign Application Priority Data
Sep. 21, 2022 (CN) 202211155461.7
Sep. 18, 2023 (CN) 202311206551.9

(51) Int. Cl. A61K 31/4985; A61K 9/20; A61K 9/28; A61K 9/48

(52) U.S. Cl. CPC A61K 31/4985; A61K 9/2009; A61K 9/2013; A61K 9/2018; A61K 9/2027; A61K 9/2054; A61K 9/2059; A61K 9/2095; A61K 9/2806; A61K 9/2893; A61K 9/4858; A61K 9/4866

(57) ABSTRACT

A pharmaceutical composition, and a preparation method therefor and the use thereof. The pharmaceutical composition contains: (4aR,8R)-3-acryloyl-11-chloro-10-(2-fluoro-6-hydroxyphenyl)-8-(2-isopropyl-4-methylpyridin-3-yl)-6-methyl-2,3,4,4a,6,8-hexahydro-1H-pyrazino[1′,2′:4,5]pyrazino[2,3-c][1,8]naphthyridin-5,7-dione or a pharmaceutically acceptable salt thereof as an active ingredient; and one or more selected from a filler, a disintegrant, a lubricant, a glidant and a binder. The pharmaceutical composition has good stability and drug dissolution, and the preparation process is simple and is suitable for industrial production.

### TECHNICAL FIELD

The present disclosure relates to the field of pharmaceutical formulations, in particular to a pharmaceutical composition, a preparation method for the pharmaceutical composition, and medical use of the pharmaceutical composition.

### BACKGROUND

Lung cancer is the most prevalent cancer worldwide. In China, lung cancer ranks first among all cancers in morbidity, and possesses the highest morbidity and mortality in China. According to the data published by the American Cancer Society in 2016, about 1.8 million people develop lung cancer annually worldwide, of which nearly 80% are non-small cell lung cancers (NSCLCs). RAS gene mutations are identified in about 32% of lung cancers, and the mutation in any one of the three major subtypes of the RAS gene (HRAS, NRAS, or KRAS) may lead to the development of tumors in humans. It has been reported that the highest mutation frequency among RAS genes was observed in the KRAS gene, and KRAS mutation was detected in 25-30% of tumors. In comparison, the rates of oncogenic mutations in NRAS and HRAS family members are much lower (8% and 3%, respectively). The most common KRAS mutations are found at residues G12 and G13 in the P-loop and residue Q61. The G12C mutation is a frequent mutation in the KRAS gene (a glycine-12 mutation to cysteine). This mutation has been found in about 13% of cancers, about 43% of lung cancers, and almost 100% of MYH-related polyposis (familial colon cancer syndrome). Therefore, the development of an inhibitor for selectively inhibiting KRAS mutations is a prospective orientation. In order to improve the inhibitory activity against KRAS mutations and reduce the inhibitory activity against wild-type KRAS, the development of novel selective inhibitors against KRAS mutants with higher activity, better selectivity, and lower toxicity has great significance.

PCT/CN2020/124226 discloses a series of substituted heterocyclic fused cyclic compounds with relatively high inhibitory activity against KRAS. A compound of formula (I), having the chemical name “(4aR,8R)-3-acryloyl-11-chloro-10-(2-fluoro-6-hydroxyphenyl)-8-(2-isopropyl-4-methylpyridin-3-yl)-6-methyl-2,3,4,4a,6,8-hexahydro-1H-pyrazino[1′,2′:4,5]pyrazino[2,3-c][1,8]naphthyridin-5,7-dione”, is described in this patent application, which has relatively high inhibitory activity against KRAS. The subsequent research and development of pharmaceutical formulations on the compound of formula (I) has important clinical significance and application prospects.

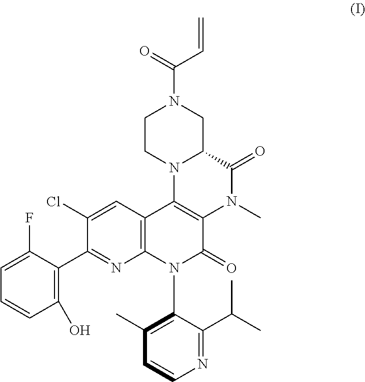

### SUMMARY

The present disclosure aims to provide a pharmaceutical composition comprising an active ingredient (4aR,8R)-3-acryloyl-11-chloro-10-(2-fluoro-6-hydroxyphenyl)-8-(2-isopropyl-4-methylpyridin-3-yl)-6-methyl-2,3,4,4a,6,8-hexahydro-1H-pyrazino[1′,2′:4,5]pyrazino[2,3-c][1,8]naphthyridin-5,7-dione or a pharmaceutically acceptable salt thereof. The pharmaceutical composition has good stability and drug dissolution, as well as better pharmacodynamic and pharmacokinetic advantages, and the preparation process is simple and suitable for industrial production.

In one aspect of the present disclosure, provided is a pharmaceutical composition comprising (4aR,8R)-3-acryloyl-11-chloro-10-(2-fluoro-6-hydroxyphenyl)-8-(2-isopropyl-4-methylpyridin-3-yl)-6-methyl-2,3,4,4a,6,8-hexahydro-1H-pyrazino[1′,2′:4,5]pyrazino[2,3-c][1,8]naphthyridin-5,7-dione or a pharmaceutically acceptable salt thereof as an active ingredient, and one or more selected from a filler, a disintegrant, a lubricant, a glidant, and a binder.

In one embodiment, the pharmaceutical composition comprises, in percentages by weight, 10%-50% of the active ingredient. For example, the percentage by weight may be 10%-40%, 10%-30%, 20%-50%, 20%-40%, 20%-30%, or 30%-40%.

Preferably, the pharmaceutical composition comprises 30%-40% of the active ingredient.

In one embodiment, the filler is selected from one or more of cellulose, silicified microcrystalline cellulose, starch, calcium hydrogen phosphate, sucrose, lactose, mannitol, xylitol, lactitol, and maltodextrin.

Preferably, the cellulose comprises microcrystalline cellulose.

Preferably, the starch comprises pregelatinized starch.

Preferably, the lactose comprises anhydrous lactose and lactose monohydrate.

More preferably, the filler is microcrystalline cellulose and lactose.

In one embodiment, the disintegrant is selected from one or more of sodium starch glycolate, sodium carboxymethyl starch, corn starch, low-substituted hydroxypropyl cellulose, crospovidone, croscarmellose sodium, croscarmellose, methylcellulose, pregelatinized starch, and sodium alginate.

Preferably, the disintegrant is croscarmellose sodium.

In one embodiment, the lubricant is selected from one or more of stearic acid, magnesium stearate, sodium stearate, calcium stearate, zinc stearate, glyceryl monostearate, glyceryl distearate, glyceryl tristearate, myristic acid, palmitic acid, sodium stearyl fumarate, talcum powder, hydrogenated vegetable oil, and mineral oil.

Preferably, the lubricant is magnesium stearate or sodium stearyl fumarate.

In one embodiment, the glidant is selected from one or more of powdered cellulose, magnesium trisilicate, colloidal silicon dioxide, silicon dioxide, and talcum powder.

Preferably, the glidant is silicon dioxide or colloidal silicon dioxide.

In one embodiment, the binder is selected from one or more of sodium carboxymethylcellulose, hydroxypropyl cellulose, hydroxypropyl methylcellulose, methylcellulose, ethylcellulose, polyvinylpyrrolidone, polyethylene glycol, povidone, copovidone, gelatin, sucrose, acacia, guar gum, and pectin.

Preferably, the binder is hydroxypropyl cellulose or copovidone.

In one embodiment, the filler is microcrystalline cellulose and lactose, wherein the lactose and the microcrystalline cellulose are in a mass ratio of (0.5-2.5):1, preferably 1:2 or 2:1.

In one embodiment, a content of the filler in percentages by weight is 10%-70%, for example, the content may be 10%-60%, 10%-50%, 10%-40%, 10%-30%, 10%-20%, 20%-70%, 20%-60%, 20%-50%, 20%-40%, 20%-30%, 30%-70%, 30%-60%, 30%-50%, 30%-40%, 40%-70%, 40%-60%, 40%-50%, 50%-70%, 50%-60%, or 60%-70%.

Preferably, the content of the filler in percentages by weight is 50%-70%.

In one embodiment, a content of the disintegrant in percentages by weight is 1%-10%, for example, the content may be 1%-4%, 1%-5%, or 4%-5%.

Preferably, the content of the disintegrant in percentages by weight is 2%-8%.

In one embodiment, a content of the lubricant in percentages by weight is 1%-5%, for example, the content may be 1%-3.5%.

Preferably, the content of the lubricant in percentages by weight is 1%-2%.

In one embodiment, a content of the glidant in percentages by weight is 0%-5%, for example, the content may be 0%-0.5%, 0%-1%, or 0%-2.5%.

In one embodiment, a content of the binder in percentages by weight is 0%-10%, for example, the content may be 0%-3%, 0%-6%, or 3%-6%.

Preferably, the content of the binder in percentages by weight is 0%-5%, which is beneficial to reducing the risk of adhering to a roller in the granulation process.

In one embodiment, the pharmaceutical composition comprises, in percentages by weight, 10%-50% of the active ingredient, 10%-70% of the filler, 1%-10% of the disintegrant, 1%-5% of the lubricant, 0%-5% of the glidant, or 0%-5% of the binder.

In one embodiment, in the pharmaceutical composition, each unit dosage form comprises 10-1000 mg of the active ingredient, for example, 50 mg, 100 mg, 150 mg, 250 mg, or 500 mg of the active ingredient.

Preferably, each unit dosage form comprises 10-500 mg of the active ingredient.

More preferably, each unit dosage form comprises 50 mg or 150 mg of the active ingredient.

In one embodiment, the pharmaceutical composition is in a form suitable for oral administration.

Preferably, the form of the pharmaceutical composition comprises a tablet or a capsule.

In one embodiment, the pharmaceutical composition is a tablet.

In one embodiment, the weight of the coating material on the surface of the tablet is 1%-5% of the weight of a plain tablet, for example, the weight of the coating material may be 1%-3% or 3%-5% of the weight of the plain tablet.

Preferably, the weight of the coating material is 2.5%-3.5% of the weight of the plain tablet.

In one embodiment, the coating material is selected from one or more of Opadry, ethylcellulose, polyvinyl alcohol, hydroxypropyl cellulose, polyethylene glycol, and polymethacrylate; preferably, the coating material is Opadry.

In another aspect of the present disclosure, provided is a preparation method for the pharmaceutical composition described above, comprising the following steps:
(1) sieving: sieving materials separately according to pharmaceutical composition formulas for later use;
(2) premixing: premixing the active ingredient, the disintegrant, and part or all of the filler obtained by the sieving in step (1);
(3) pre-lubrication: adding part of the lubricant and mixing for pre-lubrication;
(4) granulation: granulating the mixture obtained in step (3);
(5) final mixing: optionally, adding the remaining filler and performing final mixing; and
(6) final lubrication: adding the remaining lubricant, mixing, and performing final lubrication; wherein optionally, in step (2), the premixing further comprises adding the binder and part of or all of the glidant;
optionally, in step (5), the final mixing further comprises adding the remaining glidant; preferably, in step (3), the ratio of the lubricant added during the pre-lubrication to the lubricant added during the final lubrication is 1:(1-3);
preferably, the preparation method further comprising the following steps:
(7) tableting or capsule filling: tableting the mixture or filling the mixture into a capsule shell to obtain the pharmaceutical composition; and/or
(8) coating.

In one embodiment, in step (1), the sieving is performed using a 20-mesh to 100-mesh sieve.

In one embodiment, in step (2), the premixing is preferably performed at a rotation speed of 10-20 rpm; the mixing is preferably performed for a period of 10-20 min.

In one embodiment, in step (3), the pre-lubrication is preferably performed at a rotation speed of 10-20 rpm; the mixing is preferably performed for a period of 5-10 min.

In one embodiment, in step (4), the granulation process comprises feeding the mixture obtained in step (3), followed by roller pressing, sizing, and sieving, wherein the feeding is preferably performed at a speed of 3-50 rpm, the roller is preferably used at a pressure of 2-6 Mpa and preferably used at a rotation speed of 0.2-3 rpm, and the sizing is preferably performed using a 10-mesh to 20-mesh sieve; the granulation process is selected from dry granulation and wet granulation.

In the present disclosure, the feeding speed is the rotation speed of the device during feeding.

In one embodiment, in step (5), the final mixing is preferably performed at a rotation speed of 10-20 rpm; the mixing is preferably performed for a period of 5-10 min.

In one embodiment, in step (6), the final lubrication is preferably performed at a rotation speed of 10-20 rpm; the mixing is preferably performed for a period of 5-10 min.

In one embodiment, in step (7), the tableting is preferably performed with a tablet hardness of 100-200 N.

In one embodiment, in step (8), the coating preferably results in a weight gain of 1%-5% of the weight of a plain tablet.

The filler, the disintegrant, the lubricant, the glidant, or the binder may be mixed with the active ingredient by an extragranular addition method or an intragranular addition method. Specifically, the method may be selected from intragranular addition, extragranular addition, and partial intragranular and extragranular addition.

In another aspect of the present disclosure, provided is use of the pharmaceutical composition described above or a pharmaceutical composition prepared by the method described above in the preparation of a medicament for treating cancer.

In another aspect of the present disclosure, provided is a method for treating cancer, comprising the step of administering to a subject the pharmaceutical composition described above.

Preferably, the method comprises the step of administering to the subject a therapeutically effective amount of the pharmaceutical composition described above.

In one embodiment, the cancer is a KRAS G12C mutant cancer.

In one embodiment, the cancer is a solid tumor.

In one embodiment, the cancer is a solid tumor or a hematological tumor.

In one embodiment, the cancer is pancreatic ductal carcinoma, colorectal cancer, multiple myeloma, lung cancer, cutaneous melanoma, uterine corpus endometrioid carcinoma, uterine carcinosarcoma, thyroid cancer, acute myeloid leukemia, bladder urothelial carcinoma, gastric cancer, cervical cancer, head and neck squamous cell carcinoma, diffuse large B cell lymphoma, esophageal cancer, chronic lymphocytic leukemia, squamous cell lung cancer, small cell lung cancer, papillary renal cell carcinoma, adenoid cystic carcinoma, chromophobe renal cell carcinoma, liver cancer, invasive breast carcinoma, cervical squamous cell carcinoma, ovarian serous adenocarcinoma, adrenocortical carcinoma, prostate cancer, neuroblastoma, brain low-grade glioma, glioblastoma, medulloblastoma, esophageal squamous cell carcinoma, clear cell renal cell carcinoma, osteosarcoma, ovarian small cell carcinoma, rhabdomyoid tumor, sarcoma, small bowel neuroendocrine tumor, or T cell prolymphocytic leukemia.

In one embodiment, the cancer is lung adenocarcinoma, colon cancer, rectal cancer, or lung cancer.

In one embodiment, the cancer is lung adenocarcinoma, rectal adenocarcinoma, or lung cancer.

Preferably, the cancer is lung cancer (e.g., non-small cell lung cancer), pancreatic cancer, or colorectal cancer.

In one embodiment, the lung cancer is small cell lung cancer or non-small cell lung cancer.

### Beneficial Effects of Present Disclosure

1. The present disclosure provides a pharmaceutical composition formula suitable for industrial production, and the compatibility of excipients and active ingredients is intensively studied, and the qualified tablet formulation can be prepared by a preparation process of the formulation commonly used in the art, such as dry granulation.
2. The content and the proportion of excipients are further studied in the present disclosure, and the appearance properties of the obtained composition tablets in the present disclosure can meet the quality requirement through a simple process.
3. The tablets prepared in the present disclosure exhibit a smooth surface and a friability of less than 1%, further less than 0.8%, and pass the inspection. The disintegration time is less than 15 min, further less than 10 min or 6 min. The tablets prepared exhibit dissolution of greater than 75%, further greater than 85%, in the dissolution medium at 45 min, which meets the release requirement.

### DETAILED DESCRIPTION

The present disclosure will be further illustrated with reference to the following specific examples. It should be understood that these examples are merely intended to illustrate the present disclosure rather than limit the scope of the present disclosure. Unless otherwise specified, the instruments, materials, and reagents used in the following examples are available from conventional commercial sources.

### I. Definitions

Unless otherwise specified, the contents in the present disclosure are all expressed as percentages by weight, and the % content refers to weight % content.

The disclosure of all ranges in the present disclosure should be construed as disclosing all sub-ranges and all point values within the range. For example: the disclosure of 10%-70% should be construed to also disclose the ranges of 10%-50%, 50%-60%, and the like, as well as the point values of 20%, 30%, 40%, 44%, 50%, 60%, and the like.

In the present disclosure, unless otherwise specified, scientific and technical terms used herein have the meanings generally understood by those skilled in the art. In addition, the related terms and laboratory procedures used herein are the terms and conventional procedures widely used in the corresponding fields. Also, in order to better understand the present disclosure, the definitions and interpretations of the related terms are provided below.

As used herein, the term “intragranular addition” refers to the addition of materials during granulation, for example, the addition of excipients during the premixing or pre-lubrication stage in the examples of the present disclosure is intragranular addition.

As used herein, the term “extragranular addition” refers to the addition of materials to dry granulated particles prior to tableting, for example, the addition of excipients during the final mixing or final lubrication stage in the examples of the present disclosure is extragranular addition.

As used herein, the term “plain tablet” refers to an uncoated tablet obtained by pressing.

As used herein, the term “dry granulation” refers to a process in which powders of drugs and excipients are uniformly mixed, subsequently extruded into large sheets or plates, and then crushed and sized to produce the desired particles. In dry granulation, mechanical pressure is primarily utilized to reduce the distance between particles, thereby generating binding forces. If necessary, a dry binder may be added to enhance the binding forces between particles, ensuring that after the resulting particles are pressed into tablets, the hardness or friability of the tablets is qualified.

The term “unit dosage form” refers to a physically discrete unit suitable as a unit dose for a subject to be treated, wherein each unit contains a predetermined amount of active substance calculated to produce the desired therapeutic effect, optionally in combination with a suitable pharmaceutical carrier.

The unit dosage form may be a single daily therapeutic dose or one of a plurality of daily therapeutic doses (e.g., about 1 to 4 or more times daily). When a plurality of daily therapeutic doses are used, the unit dosage forms may be the same or different for each dose.

The active ingredient of the present disclosure may be used to inhibit the activity of KRAS G12C mutation. Therefore, the active ingredient of the present disclosure and a pharmaceutical composition comprising the active ingredient of the present disclosure may be used to treat or prevent a disease associated with KRAS G12C mutation, such as a cancer associated with KRAS G12C mutation. The cancer may be a solid tumor. For example, the cancer includes (but is not limited to) one or more selected from: lung cancer (e.g., non-small cell lung cancer), pancreatic cancer, colorectal cancer, and the like.

The pharmaceutical composition of the present disclosure comprises the active ingredient of the present disclosure and a pharmaceutically acceptable carrier. The pharmaceutical composition of the present disclosure may also comprise, optionally, an additional therapeutic agent. As used herein, the “pharmaceutically acceptable carrier” refers to a non-toxic, inert, solid, or semi-solid substance, a liquid filling machine, diluent, encapsulating material, or auxiliary agent, or any excipient that is compatible with the patient, preferably a mammal, more preferably a human, and suitable for delivering an active agent to a target of interest without terminating the activity of the agent.

The medicament of the present disclosure may be used alone or in combination with one or more additional therapeutic agents as desired during the course of treatment. The combination may refer to the concurrent administration of the medicament of the present disclosure and one or more additional therapeutic agents, or the administration of one or more additional therapeutic agents prior to or after the administration of the medicament of the present disclosure.

In general, the active ingredient of the present disclosure may be administered in suitable dosage forms formulated with one or more pharmaceutically acceptable carriers. Such dosage forms are suitable for oral, rectal, topical, intraoral, and other parenteral (e.g., subcutaneous, intramuscular, intravenous, etc.) administration. For example, suitable dosage forms for other parenteral administration include injections and the like. The dosage forms described above may be prepared from the active ingredient of the present disclosure and one or more carriers or excipients by conventional pharmaceutical methods. The carrier described above needs to be compatible with the active ingredient of the present disclosure or other excipients. For solid formulations, common non-toxic carriers include, but are not limited to, mannitol, lactose, starch, magnesium stearate, glucose, sucrose, and the like. The carrier for liquid formulations includes water (preferably sterile water for injection) and the like. The active ingredient of the present disclosure may be combined with the carriers described above to form a solution or a suspension.

The pharmaceutical composition of the present disclosure is formulated, quantified, and administered as per pharmaceutical practices. The “therapeutically effective amount” for administering the active ingredient of the present disclosure will be determined by such factors as the particular disorder to be treated, the individual to be treated, the cause of the disorder, the target of the medicament, and the mode of administration. As used herein, the “therapeutically effective amount” refers to an amount that produces a function or activity in a patient (e.g., a human and/or an animal) and is acceptable in humans and/or animals.

Tablets comprising (4aR,8R)-3-acryloyl-11-chloro-10-(2-fluoro-6-hydroxyphenyl)-8-(2-isopropyl-4-methylpyridin-3-yl)-6-methyl-2,3,4,4a,6,8-hexahydro-1H-pyrazino[1′,2′:4,5]pyrazino[2,3-c][1,8]naphthyridin-5,7-dione or a pharmaceutically acceptable salt thereof as an active ingredient may be coated by methods well known in the art. It should be understood that the active ingredient may further comprise polymorphs, solvates, or hydrates of (4aR,8R)-3-acryloyl-11-chloro-10-(2-fluoro-6-hydroxyphenyl)-8-(2-isopropyl-4-methylpyridin-3-yl)-6-methyl-2,3,4,4a,6,8-hexahydro-1H-pyrazino[1′,2′:4,5]pyrazino[2,3-c][1,8]naphthyridin-5,7-dione.

In the present application, the pharmaceutical composition described herein is prepared based on the active ingredient (4aR,8R)-3-acryloyl-11-chloro-10-(2-fluoro-6-hydroxyphenyl)-8-(2-isopropyl-4-methylpyridin-3-yl)-6-methyl-2,3,4,4a,6,8-hexahydro-1H-pyrazino[1′,2′:4,5]pyrazino[2,3-c][1,8]naphthyridin-5,7-dione or a pharmaceutically acceptable salt thereof, the compatibility of the excipients and the active ingredient is studied, and the changes in disintegration time, dissolution rate, or tablet hardness of different pharmaceutical compositions and the effects of the changes on product performance (e.g., bioavailability, etc.) are investigated. Also, it has surprisingly been found that the physical properties of these compositions are stable during storage. Moreover, the pharmaceutical composition prepared herein is more suitable for industrial production (e.g., reduced risk of adhering to a roller, etc.).

As used herein, the “patient” refers to an animal, preferably a mammal, more preferably a human. The term “mammal” refers to warm-blooded vertebrate mammals, including, for example, cats, dogs, rabbits, bears, foxes, wolves, monkeys, deer, mice, pigs, and humans.

As used herein, “treating” or “treatment” refers to the alleviation, delayed progression, attenuation, prevention, or maintenance of an existing disease or disorder (e.g., cancer). The “treatment” also includes the curing, prevention of development, or alleviation to some extent of one or more symptoms of a disease or disorder.

### II. Examples

### Test Method:

Dissolution determination: refer to 0931 “Dissolution and Drug Release Test”, Method 2 (Paddle), Pharmacopoeia of the People's Republic of China (2020). The dissolution was determined by using an automatic sampling dissolution tester. The temperature of a water bath of the automatic sampling dissolution tester was set to 37±0.5° C., the rotation speed was set to 75 rpm, and an acetate buffer with a pH of 4.5 containing 0.1% lauryl sodium sulfate was selected as a dissolution medium, with the volume of 900 mL. Sampling was performed at 5 min, 10 min, 20 min, 30 min, 45 min, and 60 min, respectively. All samples were filtered with a 0.45 μm filter membrane, and then determined and analyzed according to the dissolution determination method.

Friability determination: the friability of the tablets was determined with reference to 0923 “Test for Tablet Friability”, Pharmacopoeia of the People's Republic of China (2020).

Determination of disintegration time: the disintegration time of the tablets was determined with reference to 0921 “Determination of Disintegration”, Pharmacopoeia of the People's Republic of China (2020). The compound of formula (I) used in the following examples may be prepared according to the method described in Example 25 of PCT/CN2020/124226.

Unless otherwise specified, the weight percentages described in the following examples of the present disclosure are based on 100% of the total weight of the plain tablet.

### Example 1. Coated Tablet

This coated tablet contained 150 mg of the compound of formula (I) (active ingredient), 85.5 mg of microcrystalline cellulose, 178.5 mg of lactose, 18 mg of croscarmellose sodium, 2.25 mg of silicon dioxide, 15.75 mg of sodium stearyl fumarate, and 13.5 mg of Opadry.

| Component | Weight percentage |
|---|---|
| Compound of formula (I) (active ingredient) | 33.3% |
| Microcrystalline cellulose | 19% |
| Lactose | 39.7% |
| Croscarmellose sodium | 4% |
| Sodium stearyl fumarate | 3.5% |
| Silicon dioxide | 0.5% |
| Opadry | 3% |
| Total | 103% |

### Preparation Process

(1) Sieving: the compound of formula (I), microcrystalline cellulose, lactose, croscarmellose sodium, and silicon dioxide were separately sieved with a 20-mesh sieve; and sodium stearyl fumarate was sieved with a 40-mesh sieve;
(2) premixing: the compound of formula (I), microcrystalline cellulose, lactose, and croscarmellose sodium were mixed at a rotation speed of 10 rpm for 10 min;
(3) pre-lubrication: sodium stearyl fumarate (2.25 mg) was added and continued to be mixed at a rotation speed of 10 rpm for 5 min;
(4) granulation: the mixture obtained in step (3) was added to a dry granulator; the roller was used at a pressure of 2 Mpa and at a rotation speed of 1.5 rpm, the feeding was performed at a speed of 40-50 rpm, and the sizing was performed using a 20-mesh sieve;
(5) final mixing: the granulated particles obtained in step (4) and silicon dioxide were mixed at a rotation speed of 10 rpm for 10 min;
(6) final lubrication: sodium stearyl fumarate (13.5 mg) was added and continued to be mixed at a rotation speed of 10 rpm for 5 min;
(7) tableting: the final lubricated particles were subjected to tableting, with a weight of 450 mg±5% and a tablet hardness of 100-160 N; and
(8) coating: a coating powder was added to water and prepared into a 20% concentration. The plain tablet obtained by pressing in step (7) was transferred to a coating machine for coating; and the coating was terminated when the weight gain from the coating reached 3-5%.

The prepared tablet exhibited a smooth surface and a friability of less than 0.8%, and passed the inspection; the disintegration time was less than 15 min.

The prepared coated tablet exhibited dissolution (%) of greater than 85% in the dissolution medium at 45 min, which met the release requirement.

### Example 2. Common Tablet

This common tablet contained 150 mg of the compound of formula (I) (active ingredient), 178.5 mg of microcrystalline cellulose, 85.5 mg of lactose, 18 mg of croscarmellose sodium, 2.25 mg of colloidal silicon dioxide, and 15.75 mg of sodium stearyl fumarate.

| Component | Weight percentage |
|---|---|
| Compound of formula (I) (active ingredient) | 33.3% |
| Microcrystalline cellulose | 39.7% |
| Lactose | 19% |
| Croscarmellose sodium | 4% |
| Sodium stearyl fumarate | 3.5% |
| Colloidal silicon dioxide | 0.5% |
| Total | 100% |

(1) Sieving: the compound of formula (I), microcrystalline cellulose, lactose, croscarmellose sodium, and colloidal silicon dioxide were separately sieved with a 20-mesh sieve; and sodium stearyl fumarate was sieved with a 40-mesh sieve;
(2) premixing: the compound of formula (I), microcrystalline cellulose (156 mg), lactose, and croscarmellose sodium were mixed at a rotation speed of 10 rpm for 10 min;
(3) pre-lubrication: sodium stearyl fumarate (2.25 mg) was added and continued to be mixed at a rotation speed of 10 rpm for 5 min;
(4) granulation: the mixture obtained in step (3) was added to a dry granulator; the roller was used at a pressure of 2-4 Mpa and at a rotation speed of 1-2 rpm, the feeding was performed at a speed of 25-50 rpm, and the sizing was performed using a 20-mesh sieve;
(5) final mixing: the granulated particles obtained in step (4), microcrystalline cellulose (2.25 mg), and colloidal silicon dioxide were mixed at a rotation speed of 10 rpm for 10 min;
(6) final lubrication: sodium stearyl fumarate (13.5 mg) was added and continued to be mixed at a rotation speed of 10 rpm for 5 min; and
(7) tableting: the final lubricated particles were subjected to tableting, with a weight of 450 mg±5% and a tablet hardness of 100-160 N.

The prepared tablet exhibited dissolution (%) of greater than 85% in the dissolution medium at 45 min, which met the release requirement.

### Example 3. Common Tablet

This common tablet contained 150 mg of the compound of formula (I) (active ingredient), 85.5 mg of microcrystalline cellulose, 180.75 mg of lactose, 22.5 mg of croscarmellose sodium, 2.25 mg of silicon dioxide, and 9.0 mg of magnesium stearate.

| Component | Weight percentage |
|---|---|
| Compound of formula (I) (active ingredient) | 33.3% |
| Microcrystalline cellulose | 19% |
| Lactose | 40.2% |
| Croscarmellose sodium | 5% |
| Magnesium stearate | 2.0% |
| Silicon dioxide | 0.5% |
| Total | 100% |

### Preparation Process

(1) Sieving: the compound of formula (I), microcrystalline cellulose, lactose, croscarmellose sodium, and silicon dioxide were separately sieved with a 20-mesh sieve; and magnesium stearate was sieved with a 40-mesh sieve;
(2) premixing: the compound of formula (I), microcrystalline cellulose, lactose, and croscarmellose sodium were mixed at a rotation speed of 20 rpm for 10 min;
(3) pre-lubrication: magnesium stearate (2.25 mg) was added and continued to be mixed at a rotation speed of 20 rpm for 5 min;
(4) granulation: the mixture obtained in step (3) was added to a dry granulator; the roller was used at a pressure of 2 Mpa and at a rotation speed of 2 rpm, the feeding was performed at a speed of 25-50 rpm, and the sizing was performed using a 20-mesh sieve;
(5) final mixing: the dry granulated particles obtained in step (4) and silicon dioxide were mixed at a rotation speed of 20 rpm for 10 min;
(6) final lubrication: magnesium stearate (6.75 mg) was added and continued to be mixed at a rotation speed of 20 rpm for 5 min; and
(7) tableting: the tablet hardness was 100-160 N.

The disintegration time of the prepared tablet was 4-6 min; the tablet exhibited dissolution (%) of greater than 85% in the dissolution medium at 45 min, which met the release requirement.

### Example 4. Common Tablet

This common tablet contained 150 mg of the compound of formula (I) (active ingredient), 85.5 mg of microcrystalline cellulose, 178.5 mg of lactose, 18 mg of sodium carboxymethyl starch, 2.25 mg of silicon dioxide, and 15.75 mg of sodium stearyl fumarate.

| Component | Weight percentage |
|---|---|
| Compound of formula (I) (active ingredient) | 33.3% |
| Microcrystalline cellulose | 19% |
| Lactose | 39.7% |
| Sodium carboxymethyl starch | 4% |
| Sodium stearyl fumarate | 3.5% |
| Silicon dioxide | 0.5% |
| Total | 100% |

The preparation process was the same as that in Example 3 except that croscarmellose sodium was replaced by sodium carboxymethyl starch.

The disintegration time of the prepared tablet was 4-6 min; the tablet exhibited dissolution (%) of greater than 85% in the dissolution medium at 45 min, which met the release requirement.

### Example 5. Common Tablet

This common tablet contained 150 mg of the compound of formula (I) (active ingredient), 85.5 mg of microcrystalline cellulose, 151.5 mg of lactose, 45 mg of croscarmellose sodium, 2.25 mg of silicon dioxide, and 17.75 mg of sodium stearyl fumarate.

| Component | Weight percentage |
|---|---|
| Compound of formula (I) (active ingredient) | 33.3% |
| Microcrystalline cellulose | 19% |
| Lactose | 33.7% |
| Croscarmellose sodium | 10% |
| Sodium stearyl fumarate | 3.5% |
| Silicon dioxide | 0.5% |
| Total | 100% |

The preparation process was the same as that in Example 3 except that magnesium stearate was replaced by sodium stearyl fumarate, and the formula was adjusted.

The disintegration time of the prepared tablet was 4-6 min; the tablet exhibited dissolution (%) of greater than 85% in the dissolution medium at 45 min, which met the release requirement.

### Example 6. Common Tablet

This common tablet contained 150 mg of the compound of formula (I) (active ingredient), 85.5 mg of microcrystalline cellulose, 4.5 mg of silicon dioxide, 178.5 mg of lactose, 22.5 mg of croscarmellose sodium, 4.5 mg of hydroxypropyl cellulose, and 4.5 mg of magnesium stearate.

| Component | Weight percentage |
|---|---|
| Compound of formula (I) (active ingredient) | 33.3% |
| Microcrystalline cellulose | 19% |
| Silicon dioxide | 1% |
| Lactose | 39.7% |
| Croscarmellose sodium | 5% |
| Hydroxypropyl cellulose | 1% |
| Magnesium stearate | 1% |
| Total | 100% |

### Preparation Process

(1) Sieving: the compound of formula (I) was sieved with a 60-mesh sieve; microcrystalline cellulose and silicon dioxide were separately sieved with a 40-mesh sieve; and hydroxypropyl cellulose and magnesium stearate were separately sieved with a 40-mesh sieve;
(2) premixing: the compound of formula (I), microcrystalline cellulose, lactose, silicon dioxide, croscarmellose sodium, and hydroxypropyl cellulose were mixed at a rotation speed of 20 rpm for 20 min;
(3) pre-lubrication: magnesium stearate (2.25 mg) was added and continued to be mixed at a rotation speed of 20 rpm for 5 min;
(4) granulation: the mixture obtained in step (3) was added to a dry granulator; the roller was used at a pressure of 4-6 Mpa and at a rotation speed of 0.2-0.6 rpm, the feeding was performed at a speed of 3-10 rpm, and the sizing was performed using a 20-mesh sieve;
(5) final lubrication: magnesium stearate (2.25 mg) was added and continued to be mixed at a rotation speed of 20 rpm for 5 min; and
(6) tableting: the tablet hardness was 100-160 N.

The prepared tablet exhibited dissolution (%) of greater than 85% in the dissolution medium at 45 min, which met the release requirement.

### Example 7. Common Tablet

This common tablet contained 150 mg of the compound of formula (I) (active ingredient), 85.5 mg of microcrystalline cellulose, 4.5 mg of silicon dioxide, 169.5 mg of lactose, 22.5 mg of croscarmellose sodium, 13.5 mg of hydroxypropyl cellulose, and 4.5 mg of magnesium stearate.

| Component | Weight percentage |
|---|---|
| Compound of formula (I) (active ingredient) | 33.3% |
| Microcrystalline cellulose | 19% |
| Silicon dioxide | 1% |
| Lactose | 37.7% |
| Croscarmellose sodium | 5% |
| Hydroxypropyl cellulose | 3% |
| Magnesium stearate | 1% |
| Total | 100% |

The preparation process was the same as that in Example 6 except that the content of hydroxypropyl cellulose was changed to 13.5 mg, and the content of lactose was changed to 169.5 mg.

The prepared tablet exhibited dissolution (%) of greater than 85% in the dissolution medium at 45 min, which met the release requirement.

### Example 8. Common Tablet

This common tablet contained 150 mg of the compound of formula (I) (active ingredient), 85.5 mg of microcrystalline cellulose, 4.5 mg of silicon dioxide, 169.5 mg of lactose, 22.5 mg of croscarmellose sodium, 13.5 mg of copovidone, and 4.5 mg of magnesium stearate.

| Component | Weight percentage |
|---|---|
| Compound of formula (I) (active ingredient) | 33.3% |
| Microcrystalline cellulose | 19% |
| Silicon dioxide | 1% |
| Lactose | 37.7% |
| Croscarmellose sodium | 5% |
| Copovidone | 3% |
| Magnesium stearate | 1% |
| Total | 100% |

The preparation process was the same as that in Example 6 except that hydroxypropyl cellulose was replaced by copovidone, and the content of lactose was changed to 169.5 mg.

The prepared tablet exhibited dissolution (%) of greater than 85% in the dissolution medium at 45 min, which met the release requirement.

### Example 9. Common Tablet

This common tablet contained 150 mg of the compound of formula (I) (active ingredient), 85.5 mg of microcrystalline cellulose, 187.5 mg of lactose, 18 mg of croscarmellose sodium, 4.5 mg of hydroxypropyl cellulose, and 4.5 mg of sodium stearyl fumarate.

| Component | Weight percentage |
|---|---|
| Compound of formula (I) (active ingredient) | 33.3% |
| Microcrystalline cellulose | 19% |
| Lactose | 41.7% |
| Croscarmellose sodium | 4% |
| Hydroxypropyl cellulose | 1% |
| Sodium stearyl fumarate | 1% |
| Total | 100% |

### Preparation Process

(1) Sieving: the compound of formula (I), microcrystalline cellulose, lactose, and croscarmellose sodium were separately sieved with a 20-mesh sieve; hydroxypropyl cellulose and sodium stearyl fumarate were separately sieved with a 40-mesh sieve;
(2) premixing: the compound of formula (I), microcrystalline cellulose, lactose, croscarmellose sodium, and hydroxypropyl cellulose were mixed at a rotation speed of 20 rpm for 20 min;
(3) pre-lubrication: sodium stearyl fumarate (2.25 mg) was added and continued to be mixed at a rotation speed of 20 rpm for 5 min;
(4) granulation: the mixture obtained in step (3) was added to a dry granulator; the roller was used at a pressure of 4-6 Mpa and at a rotation speed of 0.2-0.6 rpm, the feeding was performed at a speed of 3-10 rpm, and the sizing was performed using a 20-mesh sieve;
(5) final lubrication: sodium stearyl fumarate (2.25 mg) was added and continued to be mixed at a rotation speed of 20 rpm for 5 min; and
(6) tableting: the tablet hardness was 100-160 N.

The prepared tablet exhibited dissolution (%) of greater than 85% in the dissolution medium at 45 min, which met the release requirement.

### Example 10. Common Tablet

This common tablet contained 150 mg of the compound of formula (I) (active ingredient), 85.5 mg of microcrystalline cellulose, 187.5 mg of lactose, 22.5 mg of croscarmellose sodium, and 4.5 mg of magnesium stearate.

| Component | Weight percentage |
|---|---|
| Compound of formula (I) (active ingredient) | 33.3% |
| Microcrystalline cellulose | 19% |
| Lactose | 41.7% |
| Croscarmellose sodium | 5% |
| Magnesium stearate | 1% |
| Total | 100% |

The preparation process was the same as that in Example 9 except that hydroxypropyl cellulose was not used in the tablet formula, magnesium stearate was replaced by sodium stearyl fumarate, and the content of croscarmellose sodium was changed to 22.5 mg.

The prepared tablet exhibited dissolution (%) of greater than 85% in the dissolution medium at 45 min, which met the release requirement.

### Example 11. Common Tablet

This common tablet contained 150 mg of the compound of formula (I) (active ingredient), 85.5 mg of microcrystalline cellulose, 192 mg of lactose, 18 mg of croscarmellose sodium, and 4.5 mg of sodium stearyl fumarate.

| Component | Weight percentage |
|---|---|
| Compound of formula (I) (active ingredient) | 33.3% |
| Microcrystalline cellulose | 19% |
| Lactose | 42.7% |
| Croscarmellose sodium | 4% |
| Sodium stearyl fumarate | 1% |
| Total | 100% |

### Preparation Process

(1) Sieving: the compound of formula (I), microcrystalline cellulose, lactose, and croscarmellose sodium were separately sieved with a 20-mesh sieve; sodium stearyl fumarate was sieved with a 40-mesh sieve;
(2) premixing: the compound of formula (I), microcrystalline cellulose, lactose, and croscarmellose sodium were mixed at a rotation speed of 10 rpm for 10 min;
(3) pre-lubrication: sodium stearyl fumarate (2.25 mg) was added and continued to be mixed at a rotation speed of 10 rpm for 5 min;
(4) granulation: the mixture obtained in step (3) was added to a dry granulator; the roller was used at a pressure of 3-6 Mpa and at a rotation speed of 1-3 rpm, the feeding was performed at a speed of 20-40 rpm, and the sizing was performed using a 20-mesh sieve;
(5) final lubrication: sodium stearyl fumarate (2.25 mg) was added and continued to be mixed at a rotation speed of 10 rpm for 5 min; and
(6) tableting: the tablet hardness was 100-160 N.

The prepared tablet had good content uniformity and exhibited dissolution (%) of greater than 85% in the dissolution medium at 45 min, which met the release requirement.

### Example 12. Common Tablet

This common tablet contained 150 mg of the compound of formula (I) (active ingredient), 85.5 mg of microcrystalline cellulose, 192 mg of lactose, 18 mg of crospovidone, and 4.5 mg of sodium stearyl fumarate.

| Component | Weight percentage |
|---|---|
| Compound of formula (I) (active ingredient) | 33.3% |
| Microcrystalline cellulose | 19% |
| Lactose | 42.7% |
| Crospovidone | 4% |
| Sodium stearyl fumarate | 1% |
| Total | 100% |

### Preparation Process

(1) Sieving: the compound of formula (I), microcrystalline cellulose, lactose, and crospovidone were separately sieved with a 20-mesh sieve; sodium stearyl fumarate was sieved with a 40-mesh sieve;
(2) premixing: the compound of formula (I), microcrystalline cellulose, lactose, and crospovidone were mixed at a rotation speed of 20 rpm for 20 min;
(3) pre-lubrication: sodium stearyl fumarate (2.25 mg) was added and continued to be mixed at a rotation speed of 20 rpm for 5 min;
(4) granulation: the mixture obtained in step (3) was added to a dry granulator; the roller was used at a pressure of 4-6 Mpa and at a rotation speed of 0.2-0.6 rpm, the feeding was performed at a speed of 3-10 rpm, the strip thickness was 1-1.5 mm, and the particles were sized and sieved with 14-mesh, 16-mesh, and 18-mesh sieves, respectively, to obtain three groups of granulated particles.
(5) final lubrication: sodium stearyl fumarate (2.25 mg) was separately added to the three groups of granulated particles obtained in step (4) and continued to be mixed at a rotation speed of 10 rpm for 5 min; and
(6) tableting: the tablet hardness was 100-160 N.

The three groups of prepared tablets all exhibited dissolution (%) of greater than 85% in the dissolution medium at 45 min, which met the release requirement.

### Example 13. Common Hard Capsule

This common hard capsule contained 150 mg of the compound of formula (I) (active ingredient), 76 mg of microcrystalline cellulose, 156 mg of lactose, 12 mg of croscarmellose sodium, and 4 mg of sodium stearyl fumarate.

| Component | Weight percentage |
|---|---|
| Compound of formula (I) (active ingredient) | 37.7% |
| Microcrystalline cellulose | 19.1% |
| Lactose | 39.2% |
| Croscarmellose sodium | 3% |
| Sodium stearyl fumarate | 1% |
| Total | 100% |

### Preparation Process

(1) Sieving: the compound of formula (I), microcrystalline cellulose, lactose, and croscarmellose sodium were separately sieved with a 20-mesh sieve; sodium stearyl fumarate was sieved with a 40-mesh sieve;
(2) premixing: the compound of formula (I), microcrystalline cellulose, lactose, and croscarmellose sodium were mixed at a rotation speed of 10 rpm for 10 min;
(3) pre-lubrication: sodium stearyl fumarate (2 mg) was added and continued to be mixed at a rotation speed of 10 rpm for 5 min;
(4) granulation: the mixture obtained in step (3) was added to a dry granulator; the roller was used at a pressure of 3-6 Mpa and at a rotation speed of 1-3 rpm, the feeding was performed at a speed of 20-40 rpm, and the sizing was performed using a 20-mesh sieve;
(5) final lubrication: sodium stearyl fumarate (2 mg) was added and continued to be mixed at a rotation speed of 10 rpm for 5 min; and
(6) capsule filling: the final mixed powder obtained in step (5) was filled into an empty gelatin capsule.

The prepared capsule exhibited dissolution (%) of greater than 85% in the dissolution medium at 45 min, which met the release requirement.

### Example 14. Common Tablet

This common tablet contained 150 mg of the compound of formula (I) (active ingredient), 120 mg of microcrystalline cellulose, 6 mg of silicon dioxide, 282 mg of lactose, 30 mg of croscarmellose sodium, 6 mg of hydroxypropyl cellulose, and 6 mg of magnesium stearate.

| Component | Weight percentage |
|---|---|
| Compound of formula (I) (active ingredient) | 25% |
| Microcrystalline cellulose | 20% |
| Silicon dioxide | 1% |
| Lactose | 47% |
| Croscarmellose sodium | 5% |
| Hydroxypropyl cellulose | 1% |
| Magnesium stearate | 1% |
| Total | 100% |

### Preparation Process

The preparation process was the same as that in Example 6 except that the contents of the active ingredient, microcrystalline cellulose, and lactose were changed.

### Example 15. Common Tablet

This common tablet contained 90 mg of the compound of formula (I) (active ingredient), 138 mg of microcrystalline cellulose, 6 mg of silicon dioxide, 282 mg of lactose, 48 mg of croscarmellose sodium, 24 mg of hydroxypropyl cellulose, and 412 mg of magnesium stearate.

| Component | Weight percentage |
|---|---|
| Compound of formula (I) (active ingredient) | 15% |
| Microcrystalline cellulose | 23% |
| Silicon dioxide | 1% |
| Lactose | 47% |
| Croscarmellose sodium | 8% |
| Hydroxypropyl cellulose | 4% |
| Magnesium stearate | 2% |
| Total | 100% |

### Preparation Process

The preparation process was the same as that in Example 6 except that the contents were changed.

### Example 16. Common Tablet

This common tablet contained 150 mg of the compound of formula (I) (active ingredient), 85.5 mg of microcrystalline cellulose, 4.5 mg of silicon dioxide, 156 mg of lactose, 22.5 mg of croscarmellose sodium, 27 mg, of hydroxypropyl cellulose, and 4.5 mg of magnesium stearate.

| Component | Weight percentage |
|---|---|
| Compound of formula (I) (active ingredient) | 33.3% |
| Microcrystalline cellulose | 19% |
| Silicon dioxide | 1% |
| Lactose | 34.7% |
| Croscarmellose sodium | 5% |
| Hydroxypropyl cellulose | 6% |
| Magnesium stearate | 1% |
| Total | 100% |

### Preparation Process

(1) Sieving: the compound of formula (I), microcrystalline cellulose, and silicon dioxide were mixed and sieved with a 40-mesh sieve; and hydroxypropyl cellulose and magnesium stearate were separately sieved with a 40-mesh sieve;
(2) premixing: the compound of formula (I), microcrystalline cellulose, silicon dioxide, lactose, croscarmellose sodium, and hydroxypropyl cellulose were mixed at a rotation speed of 20 rpm for 20 min;
(3) pre-lubrication: magnesium stearate (2.25 mg) was added and continued to be mixed at a rotation speed of 20 rpm for 5 min;
(4) granulation: the mixture obtained in step (3) was added to a dry granulator; the roller was used at a pressure of 4-6 Mpa and at a rotation speed of 0.2-0.6 rpm, the feeding was performed at a speed of 3-10 rpm, and the sizing was performed using a 20-mesh sieve;
(5) final lubrication: magnesium stearate (2.25 mg) was added and continued to be mixed at a rotation speed of 20 rpm for 5 min; and
(6) tableting: the tablet hardness was 100-160 N.

### Example 17. Common Tablet

This common tablet contained 150 mg of the compound of formula (I) (active ingredient), 85.5 mg of microcrystalline cellulose, 203.25 mg of lactose, 2.25 mg of silicon dioxide, and 9 mg of magnesium stearate.

| Component | Weight percentage |
|---|---|
| Compound of formula (I) (active ingredient) | 33.3% |
| Microcrystalline cellulose | 19% |
| Lactose | 45.2% |
| Magnesium stearate | 2% |
| Silicon dioxide | 0.5 |
| Total | 100% |

### Preparation Process

(1) Sieving: the compound of formula (I), microcrystalline cellulose, lactose, and silicon dioxide were separately sieved with a 20-mesh sieve; and magnesium stearate was sieved with a 40-mesh sieve;
(2) premixing: the compound of formula (I), microcrystalline cellulose, and lactose were mixed at a rotation speed of 10 rpm for 10 min;
(3) pre-lubrication: magnesium stearate (2.25 mg) was added and continued to be mixed at a rotation speed of 10 rpm for 5 min;
(4) granulation: the mixture obtained in step (3) was added to a dry granulator; the roller was used at a pressure of 2 Mpa and at a rotation speed of 2 rpm, the feeding was performed at a speed of 25-50 rpm, and the sizing was performed using a 20-mesh sieve;
(5) final mixing: the dry granulated particles obtained in step (4) and silicon dioxide were mixed at a rotation speed of 10 rpm for 10 min;
(6) final lubrication: magnesium stearate (6.75 mg) was added and continued to be mixed at a rotation speed of 10 rpm for 5 min; and
(7) tableting: the tablet hardness was 100-160 N.

The tablet exhibited dissolution (%) of less than 85% in the dissolution medium at 45 min, which did not meet the release requirement.

### Example 18. Common Tablet

This common tablet contained 150 mg of the compound of formula (I) (active ingredient), 85.5 mg of microcrystalline cellulose, 178.5 mg of lactose, 18 mg of croscarmellose sodium, 2.25 mg of silicon dioxide, and 15.75 mg of sodium stearyl fumarate.

| Component | Weight percentage |
|---|---|
| Compound of formula (I) (active ingredient) | 33.3% |
| Microcrystalline cellulose | 19% |
| Lactose | 39.7% |
| Croscarmellose sodium | 4% |
| Sodium stearyl fumarate | 3.5% |
| Silicon dioxide | 0.5% |
| Total | 100% |

### Preparation Process

(1) Sieving: the compound of formula (I), microcrystalline cellulose, lactose, croscarmellose sodium, and silicon dioxide were separately sieved with a 20-mesh sieve; and sodium stearyl fumarate was sieved with a 40-mesh sieve;
(2) premixing: the compound of formula (I), microcrystalline cellulose, lactose, and croscarmellose sodium were mixed at a rotation speed of 10 rpm for 10 min;
(3) pre-lubrication: sodium stearyl fumarate (2.25 mg) was added and continued to be mixed at a rotation speed of 10 rpm for 5 min;
(4) granulation: the mixture obtained in step (3) was added to a dry granulator; the roller was used at a pressure of 2 Mpa and at a rotation speed of 1.5 rpm, the feeding was performed at a speed of 40-50 rpm, and the sizing was performed using a 20-mesh sieve;
(5) final mixing: the granulated particles obtained in step (4) and silicon dioxide were mixed at a rotation speed of 10 rpm for 10 min;
(6) final lubrication: sodium stearyl fumarate (13.5 mg) was added and continued to be mixed at a rotation speed of 10 rpm for 5 min; and
(7) tableting: the final lubricated particles were subjected to tableting, with a weight of 450 mg±5% and a tablet hardness of 100-160 N; and

The prepared tablet exhibited a smooth surface and a friability of less than 0.8%, and passed the inspection; the disintegration time was less than 15 min.

The prepared coated tablet exhibited dissolution (%) of greater than 85% in the dissolution medium at 45 min, which met the release requirement.

### Example 19. Common Tablet

This common tablet contained 150 mg of the compound of formula (I) (active ingredient), 85.5 mg of microcrystalline cellulose, 183 mg of lactose, 13.5 mg of croscarmellose sodium, 2.25 mg of silicon dioxide (extragranular addition), and 15.75 mg of sodium stearyl fumarate.

| Component | Weight percentage |
|---|---|
| Compound of formula (I) (active ingredient) | 33.3% |
| Microcrystalline cellulose | 19% |
| Lactose | 40.7% |
| Croscarmellose sodium | 3% |
| Sodium stearyl fumarate | 0.5% |
| Silicon dioxide | 0.5% |
| Sodium stearyl fumarate | 3% |
| Total | 100% |

The preparation process was the same as that in Example 18 except that the content of croscarmellose sodium was changed.

### Example 20. Common Tablet

This common tablet contained 150 mg of the compound of formula (I) (active ingredient), 85.5 mg of microcrystalline cellulose, 165 mg of lactose, 31.5 mg of croscarmellose sodium, 2.25 mg of silicon dioxide, and 15.75 mg of sodium stearyl fumarate.

| Component | Weight percentage |
|---|---|
| Compound of formula (I) (active ingredient) | 33.3% |
| Microcrystalline cellulose | 19% |
| Lactose | 36.7% |
| Croscarmellose sodium | 7% |
| Sodium stearyl fumarate | 0.5% |
| Silicon dioxide | 0.5% |
| Sodium stearyl fumarate | 3% |
| Total | 100% |

The preparation process was the same as that in Example 18 except that the content of croscarmellose sodium was changed.

### Test Example 1. Stability Test

The products obtained in Example 1, Example 3, Example 10, and Example 13 were separately placed under an accelerated condition (40° C./75% RH) for 14 days and then sampling was performed to investigate the stability of the products.

TABLE 1
Stability Test Results

| | Investigation | | Content | Maximum single | Total impurity |
|---|---|---|---|---|---|
| Sample | time | | (%) | impurity (%) | (%) |
| Example 1 | 0 | days | 98.7 | 0.52 | 1.67 |
| | 30 | days | 98.2 | 0.54 | 1.72 |
| Example 3 | 0 | days | 97.9 | 0.51 | 1.70 |
| | 30 | days | 98.1 | 0.50 | 1.74 |
| Example 10 | 0 | days | 97.2 | 0.57 | 1.70 |
| | 30 | days | 97.8 | 0.54 | 1.63 |
| Example 13 | 0 | days | 98.3 | 0.56 | 1.73 |
| | 30 | days | 98.0 | 0.57 | 1.70 |

Under the condition of 40° C./75% RH described above, the contents and related substances of the samples in Example 1, Example 3, Example 10, and Example 13 were not significantly changed, and the stability of the samples was good.

### Test Example 2. Dissolution Test

The specific dissolution test results for the products in the exemplary examples of the present disclosure are shown in Table 2 below.

TABLE 2
Dissolution Test Results

| | | Dissolution (%) | | | | | |
|---|---|---|---|---|---|---|---|
| | Investigation | 5 | 10 | 20 | 30 | 45 | 60 |
| Sample | time | min | min | min | min | min | min |
| Example 1 | 0 days | 38 | 64 | 89 | 98 | 100 | 100 |
| | 30 days | 36 | 63 | 88 | 98 | 100 | 100 |
| Example 3 | 0 days | 37 | 69 | 87 | 94 | 97 | 99 |
| | 30 days | 37 | 68 | 88 | 93 | 96 | 99 |
| Example 10 | 0 days | 36 | 64 | 82 | 89 | 93 | 95 |
| | 30 days | 33 | 65 | 80 | 88 | 92 | 96 |
| Example 13 | 0 days | 38 | 69 | 89 | 96 | 97 | 98 |
| | 30 days | 35 | 63 | 86 | 97 | 98 | 98 |
| Example 17 | 0 days | 2 | 9 | 30 | 45 | 61 | 72 |
| | 30 days | 2 | 8 | 30 | 44 | 60 | 68 |

As can be seen from the data in Table 2, the tablets in Example 1, Example 3, Example 10, and Example 13 all exhibited dissolution (%) of greater than 85% at 45 min, while the tablet in Example 17 without adding a disintegrant exhibited dissolution (%) of about 6000 at 45 min, which was less than 85% and did not meet the release requirements.

### Test Example 3. Disintegration Time Test

Determination of disintegration time: the disintegration time of the tablets was determined with reference to 0921 “Determination of Disintegration”, Pharmacopoeia of the People's Republic of China (2020).

TABLE 3
Disintegration time test results

| Example No. | Disintegration time (min) |
|---|---|
| 1 | 6-10 |
| 3 | 4-6 |
| 4 | 4-6 |
| 5 | 4-6 |
| 18 | 6-10 |

As can be seen from the data in Table 3, the disintegration time of all the tablets in the exemplary examples of the present disclosure was less than 15 min, further less than 10 min or 6 min, and even reached 4-6 min, exhibiting relatively good disintegration performance.

All documents mentioned in the present disclosure are incorporated herein by reference, just as each document is individually cited as a reference. In addition, it should be understood that various modifications or changes may be made by those skilled in the art after reading the above teachings of the present disclosure, and these equivalent forms also fall within the scope defined by the appended claims of the present application.

1. A pharmaceutical composition, comprising (4aR,8R)-3-acryloyl-11-chloro-10-(2-fluoro-6-hydroxyphenyl)-8-(2-isopropyl-4-methylpyridin-3-yl)-6-methyl-2,3,4,4a,6,8-hexahydro-1H-pyrazino[1′,2′:4,5]pyrazino[2,3-c][1,8]naphthyridin-5,7-dione or a pharmaceutically acceptable salt thereof as an active ingredient, and one or more selected from a filler, a disintegrant, a lubricant, a glidant, and a binder.

2. The pharmaceutical composition according to claim 1, wherein the pharmaceutical composition comprises, in percentages by weight, 10%-50% of the active ingredient, preferably 30%-40% of the active ingredient.

3. The composition according to claim 1, wherein
the filler is selected from one or more of cellulose, silicified microcrystalline cellulose, starch, calcium hydrogen phosphate, sucrose, lactose, mannitol, xylitol, lactitol, and maltodextrin;
preferably, the cellulose comprises microcrystalline cellulose; preferably, the starch comprises pregelatinized starch; preferably, the lactose comprises anhydrous lactose and lactose monohydrate; more preferably, the filler is microcrystalline cellulose and lactose;
and/or the disintegrant is selected from one or more of sodium starch glycolate, sodium carboxymethyl starch, corn starch, low-substituted hydroxypropyl cellulose, crospovidone, croscarmellose sodium, croscarmellose, methylcellulose, pregelatinized starch, and sodium alginate; more preferably, the disintegrant is croscarmellose sodium;
and/or the lubricant is selected from one or more of stearic acid, magnesium stearate, sodium stearate, calcium stearate, zinc stearate, glyceryl monostearate, glyceryl distearate, glyceryl tristearate, myristic acid, palmitic acid, sodium stearyl fumarate, talcum powder, hydrogenated vegetable oil, and mineral oil; more preferably, the lubricant is magnesium stearate or sodium stearyl fumarate;
and/or the glidant is selected from one or more of powdered cellulose, magnesium trisilicate, colloidal silicon dioxide, silicon dioxide, and talcum powder; more preferably, the glidant is silicon dioxide or colloidal silicon dioxide;
and/or the binder is selected from one or more of sodium carboxymethylcellulose, hydroxypropyl cellulose, hydroxypropyl methylcellulose, methylcellulose, ethylcellulose, polyvinylpyrrolidone, polyethylene glycol, povidone, copovidone, gelatin, sucrose, acacia, guar gum, and pectin; more preferably, the binder is hydroxypropyl cellulose or copovidone.

4. The pharmaceutical composition according to claim 3, wherein the filler is microcrystalline cellulose and lactose, wherein the lactose and the microcrystalline cellulose are in a mass ratio of (0.5-2.5):1, preferably 1:2 or 2:1.

5. The pharmaceutical composition according to claim 3, wherein
a content of the filler in percentages by weight is 10%-70%, more preferably 50%-70%;
and/or a content of the disintegrant in percentages by weight is 1%-10%, more preferably 2%-8%;
and/or a content of the lubricant in percentages by weight is 1%-5%, more preferably 1%-3.5%;
and/or a content of the glidant in percentages by weight is 0%-5%;
and/or a content of the binder in percentages by weight is 0%-10%, more preferably 0%-5%.

6. The pharmaceutical composition according to claim 1, wherein the pharmaceutical composition comprises, in percentages by weight, 10%-50% of the active ingredient, 10%-70% of the filler, 1%-10% of the disintegrant, 1%-5% of the lubricant, 0%-5% of the glidant, and 0%-5% of the binder.

7. The pharmaceutical composition according to claim 1, wherein each unit dosage form comprises 10-1000 mg of the active ingredient, preferably 10-500 mg of the active ingredient, and more preferably 50 mg or 150 mg of the active ingredient.

8. The pharmaceutical composition according to claim 1, wherein the pharmaceutical composition is in a form suitable for oral administration;
preferably, the form of the pharmaceutical composition comprises a tablet or a capsule.

9. The pharmaceutical composition claim 1, wherein the pharmaceutical composition is a tablet;
preferably, the tablet has a coating material on the surface;
preferably, the weight of the coating material on the surface of the tablet is 1%-5% of the weight of a plain tablet, more preferably 2.5%-3.5% of the weight of the plain tablet;
preferably, the coating material is selected from one or more of Opadry, ethylcellulose, polyvinyl alcohol, hydroxypropyl cellulose, polyethylene glycol, and polymethacrylate; more preferably, the coating material is Opadry.

10. A preparation method for the pharmaceutical composition according to claim 1, comprising the following steps:
(1) sieving: sieving materials separately according to pharmaceutical composition formulas for later use;
(2) premixing: premixing the active ingredient, the disintegrant, and part or all of the filler obtained by the sieving in step (1);
(3) pre-lubrication: adding part of the lubricant and mixing for pre-lubrication;
(4) granulation: granulating the mixture obtained in step (3);
(5) final mixing: optionally, adding the remaining filler and performing final mixing; and
(6) final lubrication: adding the remaining lubricant, mixing, and performing final lubrication;
wherein optionally, in step (2), the premixing further comprises adding the binder and part of or all of the glidant;
optionally, in step (5), the final mixing further comprises adding the remaining glidant;
preferably, in step (3), the ratio of the lubricant added during the pre-lubrication to the lubricant added during the final lubrication is 1:(1-3);
preferably, the preparation method further comprising the following steps:
(7) tableting or capsule filling: tableting the mixture or filling the mixture into a capsule shell to obtain the pharmaceutical composition; and/or
(8) coating.

11. The preparation method for the pharmaceutical composition according to claim 10, wherein:
in step (1), the sieving is performed using a 20-mesh to 100-mesh sieve;
in step (2), the premixing is preferably performed at a rotation speed of 10-20 rpm; the mixing is preferably performed for a period of 10-20 min;
in step (3), the pre-lubrication is preferably performed at a rotation speed of 10-20 rpm; the mixing is preferably performed for a period of 5-10 min;
in step (4), the granulation process comprises feeding the mixture obtained in step (3), followed by roller pressing, sizing, and sieving, wherein the feeding is preferably performed at a speed of 3-50 rpm, the roller is preferably used at a pressure of 2-6 Mpa and preferably used at a rotation speed of 0.2-3 rpm, and the sizing is preferably performed using a 10-mesh to 20-mesh sieve; the granulation process is selected from dry granulation and wet granulation;
in step (5), the final mixing is preferably performed at a rotation speed of 10-20 rpm; the mixing is preferably performed for a period of 5-10 min;
in step (6), the final lubrication is preferably performed at a rotation speed of 10-20 rpm; the mixing is preferably performed for a period of 5-10 min;
in step (7), the tableting is preferably performed with a tablet hardness of 100-200 N;
in step (8), the coating preferably results in a weight gain of 1%-5% of the weight of a plain tablet.

12. (canceled)

13. A method for treating cancer, comprising the step of administering to a patient in need thereof the pharmaceutical composition according to claim 1.

14. The method according to claim 13, wherein the cancer is a KRAS G12C mutant cancer; preferably, the cancer is a solid tumor or a hematological tumor; preferably, the cancer is pancreatic ductal carcinoma, colorectal cancer, multiple myeloma, lung cancer, cutaneous melanoma, uterine corpus endometrioid carcinoma, uterine carcinosarcoma, thyroid cancer, acute myeloid leukemia, bladder urothelial carcinoma, gastric cancer, cervical cancer, head and neck squamous cell carcinoma, diffuse large B cell lymphoma, esophageal cancer, chronic lymphocytic leukemia, squamous cell lung cancer, small cell lung cancer, papillary renal cell carcinoma, adenoid cystic carcinoma, chromophobe renal cell carcinoma, liver cancer, invasive breast carcinoma, cervical squamous cell carcinoma, ovarian serous adenocarcinoma, adrenocortical carcinoma, prostate cancer, neuroblastoma, brain low-grade glioma, glioblastoma, medulloblastoma, esophageal squamous cell carcinoma, clear cell renal cell carcinoma, osteosarcoma, ovarian small cell carcinoma, rhabdomyoid tumor, sarcoma, small bowel neuroendocrine tumor, or T cell prolymphocytic leukemia; preferably, the cancer is lung adenocarcinoma, colon cancer, rectal cancer, or lung cancer; preferably, the lung cancer is small cell lung cancer or non-small cell lung cancer.

15. A method for treating cancer, comprising the step of administering to a patient in need thereof the pharmaceutical composition according to claim 2;
wherein the cancer is a KRAS G12C mutant cancer; preferably, the cancer is a solid tumor or a hematological tumor; preferably, the cancer is pancreatic ductal carcinoma, colorectal cancer, multiple myeloma, lung cancer, cutaneous melanoma, uterine corpus endometrioid carcinoma, uterine carcinosarcoma, thyroid cancer, acute myeloid leukemia, bladder urothelial carcinoma, gastric cancer, cervical cancer, head and neck squamous cell carcinoma, diffuse large B cell lymphoma, esophageal cancer, chronic lymphocytic leukemia, squamous cell lung cancer, small cell lung cancer, papillary renal cell carcinoma, adenoid cystic carcinoma, chromophobe renal cell carcinoma, liver cancer, invasive breast carcinoma, cervical squamous cell carcinoma, ovarian serous adenocarcinoma, adrenocortical carcinoma, prostate cancer, neuroblastoma, brain low-grade glioma, glioblastoma, medulloblastoma, esophageal squamous cell carcinoma, clear cell renal cell carcinoma, osteosarcoma, ovarian small cell carcinoma, rhabdomyoid tumor, sarcoma, small bowel neuroendocrine tumor, or T cell prolymphocytic leukemia; preferably, the cancer is lung adenocarcinoma, colon cancer, rectal cancer, or lung cancer; preferably, the lung cancer is small cell lung cancer or non-small cell lung cancer.

16. A method for treating cancer, comprising the step of administering to a patient in need thereof the pharmaceutical composition according to claim 3;
wherein the cancer is a KRAS G12C mutant cancer; preferably, the cancer is a solid tumor or a hematological tumor; preferably, the cancer is pancreatic ductal carcinoma, colorectal cancer, multiple myeloma, lung cancer, cutaneous melanoma, uterine corpus endometrioid carcinoma, uterine carcinosarcoma, thyroid cancer, acute myeloid leukemia, bladder urothelial carcinoma, gastric cancer, cervical cancer, head and neck squamous cell carcinoma, diffuse large B cell lymphoma, esophageal cancer, chronic lymphocytic leukemia, squamous cell lung cancer, small cell lung cancer, papillary renal cell carcinoma, adenoid cystic carcinoma, chromophobe renal cell carcinoma, liver cancer, invasive breast carcinoma, cervical squamous cell carcinoma, ovarian serous adenocarcinoma, adrenocortical carcinoma, prostate cancer, neuroblastoma, brain low-grade glioma, glioblastoma, medulloblastoma, esophageal squamous cell carcinoma, clear cell renal cell carcinoma, osteosarcoma, ovarian small cell carcinoma, rhabdomyoid tumor, sarcoma, small bowel neuroendocrine tumor, or T cell prolymphocytic leukemia; preferably, the cancer is lung adenocarcinoma, colon cancer, rectal cancer, or lung cancer; preferably, the lung cancer is small cell lung cancer or non-small cell lung cancer.

17. A method for treating cancer, comprising the step of administering to a patient in need thereof the pharmaceutical composition according to claim 6;
wherein the cancer is a KRAS G12C mutant cancer; preferably, the cancer is a solid tumor or a hematological tumor; preferably, the cancer is pancreatic ductal carcinoma, colorectal cancer, multiple myeloma, lung cancer, cutaneous melanoma, uterine corpus endometrioid carcinoma, uterine carcinosarcoma, thyroid cancer, acute myeloid leukemia, bladder urothelial carcinoma, gastric cancer, cervical cancer, head and neck squamous cell carcinoma, diffuse large B cell lymphoma, esophageal cancer, chronic lymphocytic leukemia, squamous cell lung cancer, small cell lung cancer, papillary renal cell carcinoma, adenoid cystic carcinoma, chromophobe renal cell carcinoma, liver cancer, invasive breast carcinoma, cervical squamous cell carcinoma, ovarian serous adenocarcinoma, adrenocortical carcinoma, prostate cancer, neuroblastoma, brain low-grade glioma, glioblastoma, medulloblastoma, esophageal squamous cell carcinoma, clear cell renal cell carcinoma, osteosarcoma, ovarian small cell carcinoma, rhabdomyoid tumor, sarcoma, small bowel neuroendocrine tumor, or T cell prolymphocytic leukemia; preferably, the cancer is lung adenocarcinoma, colon cancer, rectal cancer, or lung cancer; preferably, the lung cancer is small cell lung cancer or non-small cell lung cancer.

18. A method for treating cancer, comprising the step of administering to a patient in need thereof the pharmaceutical composition according to claim 7;
wherein the cancer is a KRAS G12C mutant cancer; preferably, the cancer is a solid tumor or a hematological tumor; preferably, the cancer is pancreatic ductal carcinoma, colorectal cancer, multiple myeloma, lung cancer, cutaneous melanoma, uterine corpus endometrioid carcinoma, uterine carcinosarcoma, thyroid cancer, acute myeloid leukemia, bladder urothelial carcinoma, gastric cancer, cervical cancer, head and neck squamous cell carcinoma, diffuse large B cell lymphoma, esophageal cancer, chronic lymphocytic leukemia, squamous cell lung cancer, small cell lung cancer, papillary renal cell carcinoma, adenoid cystic carcinoma, chromophobe renal cell carcinoma, liver cancer, invasive breast carcinoma, cervical squamous cell carcinoma, ovarian serous adenocarcinoma, adrenocortical carcinoma, prostate cancer, neuroblastoma, brain low-grade glioma, glioblastoma, medulloblastoma, esophageal squamous cell carcinoma, clear cell renal cell carcinoma, osteosarcoma, ovarian small cell carcinoma, rhabdomyoid tumor, sarcoma, small bowel neuroendocrine tumor, or T cell prolymphocytic leukemia; preferably, the cancer is lung adenocarcinoma, colon cancer, rectal cancer, or lung cancer; preferably, the lung cancer is small cell lung cancer or non-small cell lung cancer.

19. A method for treating cancer, comprising the step of administering to a patient in need thereof the pharmaceutical composition according to claim 9;
wherein the cancer is a KRAS G12C mutant cancer; preferably, the cancer is a solid tumor or a hematological tumor; preferably, the cancer is pancreatic ductal carcinoma, colorectal cancer, multiple myeloma, lung cancer, cutaneous melanoma, uterine corpus endometrioid carcinoma, uterine carcinosarcoma, thyroid cancer, acute myeloid leukemia, bladder urothelial carcinoma, gastric cancer, cervical cancer, head and neck squamous cell carcinoma, diffuse large B cell lymphoma, esophageal cancer, chronic lymphocytic leukemia, squamous cell lung cancer, small cell lung cancer, papillary renal cell carcinoma, adenoid cystic carcinoma, chromophobe renal cell carcinoma, liver cancer, invasive breast carcinoma, cervical squamous cell carcinoma, ovarian serous adenocarcinoma, adrenocortical carcinoma, prostate cancer, neuroblastoma, brain low-grade glioma, glioblastoma, medulloblastoma, esophageal squamous cell carcinoma, clear cell renal cell carcinoma, osteosarcoma, ovarian small cell carcinoma, rhabdomyoid tumor, sarcoma, small bowel neuroendocrine tumor, or T cell prolymphocytic leukemia; preferably, the cancer is lung adenocarcinoma, colon cancer, rectal cancer, or lung cancer; preferably, the lung cancer is small cell lung cancer or non-small cell lung cancer.

20. A method for treating cancer, comprising the step of administering to a patient in need thereof a pharmaceutical composition prepared by the method according to claim 10;
wherein the cancer is a KRAS G12C mutant cancer; preferably, the cancer is a solid tumor or a hematological tumor; preferably, the cancer is pancreatic ductal carcinoma, colorectal cancer, multiple myeloma, lung cancer, cutaneous melanoma, uterine corpus endometrioid carcinoma, uterine carcinosarcoma, thyroid cancer, acute myeloid leukemia, bladder urothelial carcinoma, gastric cancer, cervical cancer, head and neck squamous cell carcinoma, diffuse large B cell lymphoma, esophageal cancer, chronic lymphocytic leukemia, squamous cell lung cancer, small cell lung cancer, papillary renal cell carcinoma, adenoid cystic carcinoma, chromophobe renal cell carcinoma, liver cancer, invasive breast carcinoma, cervical squamous cell carcinoma, ovarian serous adenocarcinoma, adrenocortical carcinoma, prostate cancer, neuroblastoma, brain low-grade glioma, glioblastoma, medulloblastoma, esophageal squamous cell carcinoma, clear cell renal cell carcinoma, osteosarcoma, ovarian small cell carcinoma, rhabdomyoid tumor, sarcoma, small bowel neuroendocrine tumor, or T cell prolymphocytic leukemia; preferably, the cancer is lung adenocarcinoma, colon cancer, rectal cancer, or lung cancer; preferably, the lung cancer is small cell lung cancer or non-small cell lung cancer.

21. A method for treating cancer, comprising the step of administering to a patient in need thereof a pharmaceutical composition prepared by the method according to claim 11;
wherein the cancer is a KRAS G12C mutant cancer; preferably, the cancer is a solid tumor or a hematological tumor; preferably, the cancer is pancreatic ductal carcinoma, colorectal cancer, multiple myeloma, lung cancer, cutaneous melanoma, uterine corpus endometrioid carcinoma, uterine carcinosarcoma, thyroid cancer, acute myeloid leukemia, bladder urothelial carcinoma, gastric cancer, cervical cancer, head and neck squamous cell carcinoma, diffuse large B cell lymphoma, esophageal cancer, chronic lymphocytic leukemia, squamous cell lung cancer, small cell lung cancer, papillary renal cell carcinoma, adenoid cystic carcinoma, chromophobe renal cell carcinoma, liver cancer, invasive breast carcinoma, cervical squamous cell carcinoma, ovarian serous adenocarcinoma, adrenocortical carcinoma, prostate cancer, neuroblastoma, brain low-grade glioma, glioblastoma, medulloblastoma, esophageal squamous cell carcinoma, clear cell renal cell carcinoma, osteosarcoma, ovarian small cell carcinoma, rhabdomyoid tumor, sarcoma, small bowel neuroendocrine tumor, or T cell prolymphocytic leukemia; preferably, the cancer is lung adenocarcinoma, colon cancer, rectal cancer, or lung cancer; preferably, the lung cancer is small cell lung cancer or non-small cell lung cancer.
